(19) United States
(12) Patent Application Publication
(10) Pub. No.: US 2016/0157897 A1
(43) Pub. Date: Jun. 9, 2016

(54) Method for Minimally Invasive Treatment of Unstable Pelvic Ring Injuries with an Internal Posterior Iliosacral Screw and Bone Plate

(71) Applicant: Rahul Vaidya, Ann Arbor, MI (US)

(72) Inventor: Rahul Vaidya, Ann Arbor, MI (US)

(21) Appl. No.: 14/562,710

(22) Filed: Dec. 6, 2014

(51) Int. Cl. A61B 17/70; A61B 17/80

(52) U.S. Cl. CPC A61B 17/7059; A61B 17/8066

(57) ABSTRACT

The instant invention is a novel method for stabilization of the posterior pelvis (iliosacral) when treating an unstable pelvic ring fracture. Cannulated screws are inserted through the posterior of the ilium through the sacroiliac joint and into the sacrum. The screws are used to affix the iliosacral joint while it heals. The screws are used in conjunction with a bone plate (through which the screws pass) to provide the construct/fixation with added strength and stability compared to iliosacral fixation using screws alone.

### FIELD OF THE INVENTION

The instant invention relates generally to methods and apparatus for the treatment of unstable pelvic fractures. More specifically the invention relates to a method and apparatus for minimally invasive treatment of unstable pelvic ring injuries using an internal posterior iliosacral screw and bone plate construct.

### BACKGROUND OF THE INVENTION

Unstable pelvic fractures typically occur as a result of high-energy injuries such as automobile accidents, falls and the like. Even in this age of modern polytrauma care, acute pelvic fractures are potentially lethal. In the past, such injuries were treated without surgery. However, recovery to completely normal functionality was the exception rather than the norm. In more modern times, unstable pelvic fractures are treated surgically with a number of techniques depending on the type and extent of the fracture(s).

The pelvis consists of three major bones (two ilium 1, 1′ and the sacrum 2, the sacroiliac joints 3, 3′ (being where the ilia attach to the sacrum) and some minor bones joined together in a ring shape and held by strong ligaments, See FIG. 1. General characteristics of pelvic fracture include severe pain, pelvic bone instability, and associated internal bleeding. Devices and methods used to treat fracture of the pelvis currently fall under two general classifications; internal fixation and external fixation. Combinations of both techniques are frequently chosen for certain fracture patterns.

Internal fixation is typically utilized when the patient exhibits unstable posterior pelvic fractures. Internal fixation refers to plates and screws applied directly onto the fracture sites after realignment. See, for example, U.S. Pat. Nos. 4,454,876; 5,108,397; 6,340,362 and 6,440,131. This type of fracture tends to be more complex, involving multiple bony structures. Internal fixation addresses these clinical issues through open reduction and correction of misaligned bone segments that are subsequently stabilized with a wide variety of plate and screw methods.

Anterior pelvic fractures or hemodynamically unstable patients are candidates for external fixation. Pelvic external fixation consists of pins usually inserted into the iliac bones and then connected together by clamps and bars. See, for example, U.S. Pat. Nos. 4,292,964; 4,361,144; 5,350,378 and 6,162,222. External fixation methods consists of stabilizing the pelvic ring with a rigid framework residing outside the patient's body that is connected to the patient's pelvis via multiple pins that penetrate through the patient's soft and hard tissues. Several frame types are currently utilized. Two of the more widely deployed devices for external pelvic stabilization are the Hoffmann 2 Inverted “A” Frame and the Ganz Pelvic C Clamp.

The application of external reduction and fixation for pelvic fractures is advantageous compared to internal reduction and fixation due to its speed of deployment and lower level of technical training required for utilization. The primary disadvantages of external fixation of pelvic fractures include high risk of pin tract infections, and general patient discomfort. Also, the external frame physically blocks subsequent surgery on the abdomen and they are frequently difficult to fit to obese patients.

The instant inventor has previously developed novel methods using the already established principles of anterior external fixation. See U.S. Pat. Nos. 8,900,278; 8,814,866; 8,398,635; and 8,177,785, the disclosures of which are herein incorporated by reference. By combining these principles with internal hardware placed in a minimally invasive fashion, this technique allows for definitive pelvic stabilization without having the issues and co-morbidities of an external fixator (i.e. interfering with other procedures, pin care, patient acceptance, later conversion to internal fixation, etc.) The present invention adds to the internal anterior fixation pelvic stabilization using a plate and screw structure on the posterior of the pelvis.

### SUMMARY OF THE INVENTION

The present invention is a novel surgical method and apparatus for minimally invasive affixation of an ilium to the sacrum in an unstable pelvic ring injury. The method may comprise the step of providing a bone plate having at least two attachment holes therethrough, and two cannulated screws. The method may comprise the step of affixing the bone plate to the posterior of the ilium by the steps of: placing the bone plate on the ilium, adjacent to the sacroiliac joint; screwing the first of the two cannulated screws through a first of the attachment holes in the bone plate, through the ilium, through the sacroiliac joint, and into the S1 vertebral body; and screwing the second of the two cannulated screws through a second of the attachment holes in the bone plate, through the ilium, through the sacroiliac joint, and into the S2 vertebral body. The cannulated screws may be inserted far enough to hold the bone plate tightly against the ilium.

The step of placing the bone plate on the ilium, adjacent to the sacroiliac joint may comprise the steps of: inserting a first cannulation guide wire through the ilium, the sacroiliac joint, and into the S1 vertebral body; inserting a second cannulation guide wire through the ilium, the sacroiliac joint, and into the S2 vertebral body; sliding the first and second cannulation guide wires through the first and second attachment holes in the bone plate, respectively; and advancing the bone plate down along the guide wires to the surface of the ilium.

The step of screwing the first of the two cannulated screws through the first of the attachment holes in the bone plate may comprise: placing the first of the two cannulated screws onto the first cannulation guide wire; guiding the first of the two cannulated screws down the first cannulation guide wire to the surface of the ilium; and screwing the first of the two cannulated screws through the ilium, through the sacroiliac joint and into the S1 vertebral body. The first of the two cannulated screws may be of a length to reach from the bone plate to a medial position in the S1 vertebral body.

The step of screwing the second of the two cannulated screws through the second of the attachment holes in the bone plate may comprise: placing the second of the two cannulated screws onto the second cannulation guide wire; guiding the second of the two cannulated screws down the second cannulation guide wire to the surface of the ilium; and screwing the second of the two cannulated screws through the ilium, through the sacroiliac joint and into the S2 vertebral body. The second of the two cannulated screws is of a length to reach from the bone plate to through the S2 vertebral body and into the opposite ilium. The surgical method may comprise the further step of removing the first and second cannulation guide wires from the pelvis after insertion of the first and second cannulated screws.

The surgical method may comprise the further steps of: placing a second bone plate having at least two attachment holes therethrough on the opposite ilium, adjacent to the opposite sacroiliac joint; screwing the a third cannulated screw through a first of the attachment holes in the second bone plate, through the opposite ilium, through the opposite sacroiliac joint, and into the S1 vertebral body; and passing the second cannulated screw through a second of the attachment holes in the second bone plate.

The surgical method may further comprise the step of screwing threads of the forward end of the second cannulated screw into matching threads in the second of the attachment holes in the second bone plate, thereby locking the second bone plate to the second cannulated screw. Alternatively, the surgical method may comprise the further steps of: passing the forward end of the second cannulated screw through the second of the attachment holes in the second bone plate such that it protrudes from the second of the attachment holes in the second bone plate; and placing a nut onto the threads of the forward end of the second cannulated screw and tightening the nut to thereby lock the second bone plate to the second cannulated screw.

The two cannulated screws may be cancellous screws, that are fully or partially threaded. The first of the two cannulated screws may be a partially threaded cancellous screw. The second of the two cannulated screws may be a fully threaded cancellous screw. The bone plate may have two attachment holes therethrough, one at either end thereof. The second bone plate may also have two attachment holes therethrough, one at either end thereof. The bone plate may be a variable-angle locking plate and the second bone plate may be a variable-angle locking plate. The bone plate and the cannulated screws may be formed from titanium. The cannulated screws may be between 7.0 and 8.5 mm in outer diameter, inclusive. The first and second cannulation guide wires may be about 3 mm in outer diameter.

### BRIEF DESCRIPTION OF THE DRAWINGS

FIG. 1 is a depiction of a pelvis indicating the ilia, the sacrum and the sacroiliac joints;

FIG. 2A depicts the medial aspect of a left ilium;

FIG. 2B depicts the anterior view of a sacrum;

FIG. 3A is a depiction of the posterior of a pelvis having a pelvic ring fracture;

FIG. 3B is a depiction of the posterior of a pelvis, wherein the first step of the inventive method has been performed;

FIG. 3B′ depicts an anterior view of a horizontal cross section of the pelvis at the end of the first step of the inventive method has been performed;

FIG. 3C is a depiction of the posterior of a pelvis, wherein the second step of the inventive method has been performed;

FIG. 3D is a depiction of the posterior of a pelvis, wherein the third step of the inventive method has been performed;

FIG. 3D′ depicts an anterior view of a horizontal cross section of the pelvis at the end of the third step of the inventive method;

FIG. 3E is a depiction of the posterior of a pelvis, wherein the fourth step of the inventive method has been performed;

FIG. 3E′ depicts an anterior view of a horizontal cross section of the pelvis at the end of the fourth step of the inventive method;

FIG. 3F is a depiction of the posterior of a pelvis, wherein the fifth step of the inventive method has been performed;

FIG. 3G is a depiction of the posterior of a pelvis, wherein the sixth step of the inventive method has been performed;

FIG. 3G′ depicts an anterior view of a horizontal cross section of the pelvis at the end of the sixth step of the inventive method;

FIG. 3H is a depiction of the posterior of a pelvis, wherein optional additional steps have been performed to add an additional iliosacral screw and bone plate to the opposite iliosacral joint of the pelvis;

FIG. 3H′ depicts an anterior view of a horizontal cross section of the pelvis at the end of the optional additional steps of the inventive method;

FIG. 4A depicts a cross section of a cannulated screw and specifically shows how a cannulated screw may be inserted onto the cannulation guide wire;

FIG. 4B show a cannulated screw inserted onto a cannulation guide wire;

FIG. 5 is a depiction of a lateral view of a pelvis (ilium 1, sacrum 2) having been fixated using the inventive method and apparatus; and

FIGS. 6A and 6B are depictions of different views of a type bone plating system, a variable-angle locked plating system, useful in the present invention.

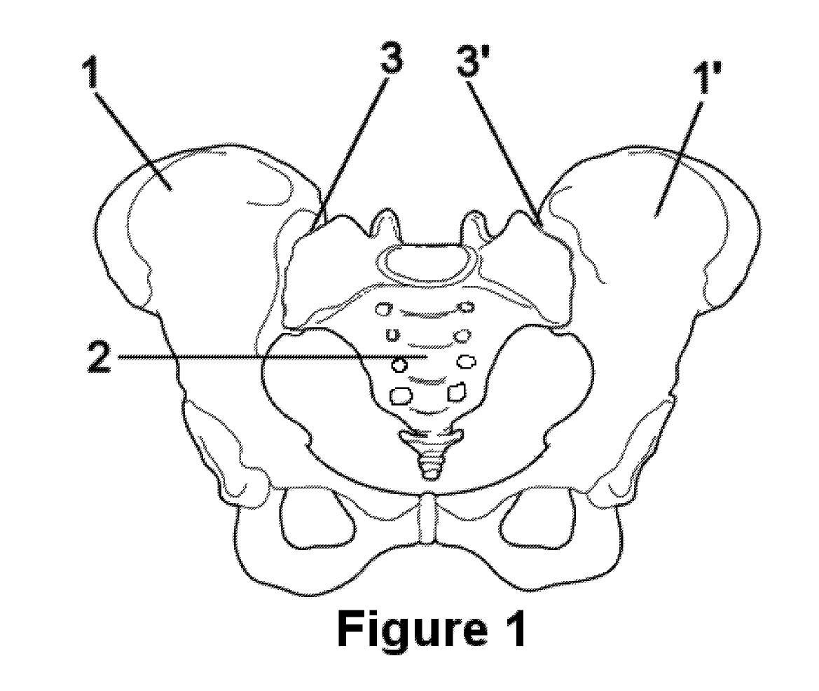

### DETAILED DESCRIPTION OF THE INVENTION

The instant invention is a novel method for posterior pelvic stabilization. The method uses internal hardware placed in a minimally invasive fashion. Stabilization of pelvic ring injuries is most often indicated when the volume of the pelvis is increased and/or an unstable pattern of injury is present. This stabilization method must be applied in the operating room under sterile conditions with adequate fluoroscopic guidance. It can be utilized in an emergent setting following provisional stabilization in the emergency room with a pelvic binder, sheet or clamp.

To aid in the determination of utilizing internal fixation methods, we prefer the Tile classification since it is based on the concept of pelvic stability. In the Tile classification, type A fractures involve a stable pelvic ring. The partially stable type B lesions, such as “open-book” and “bucket-handle” fractures, are caused by external and internal rotation forces, respectively. In type C injuries, there is complete disruption of the posterior sacroiliac complex. These unstable fractures are almost always caused by high-energy severe trauma associated with motor vehicle accidents, falls from a height, or crushing injuries. Type A and type B fractures make up 70% to 80% of all pelvic injuries. Internal fixation methods are typically considered for Tile B and C type injuries. In many patients with partially stable injury patterns, the presence of significant pain with upright posture can be alleviated with the addition of internal fixation. If adequate reduction cannot be obtained in a closed manner, then more traditional open reduction techniques need to be employed.

### Surgical Technique

The patient may be positioned in the supine position on a radiolucent table. The skin may be prepped and draped from above the umbilicus to the proximal thigh. The lower extremity may be prepped into the field as well to facilitate reduction techniques.

The posterior instability may be addressed first. The inventive procedure for placement of iliosacral screws and bone plate(s) for posterior pelvic instability will be described herein below, but first we need to describe the sacrum and sacroiliac joint in more detail.

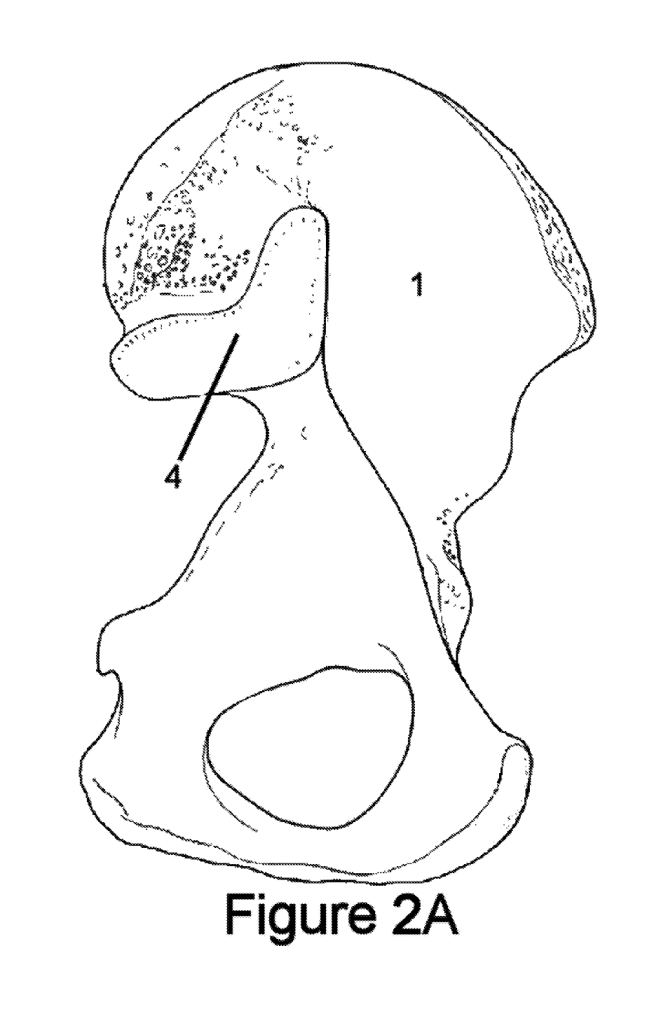

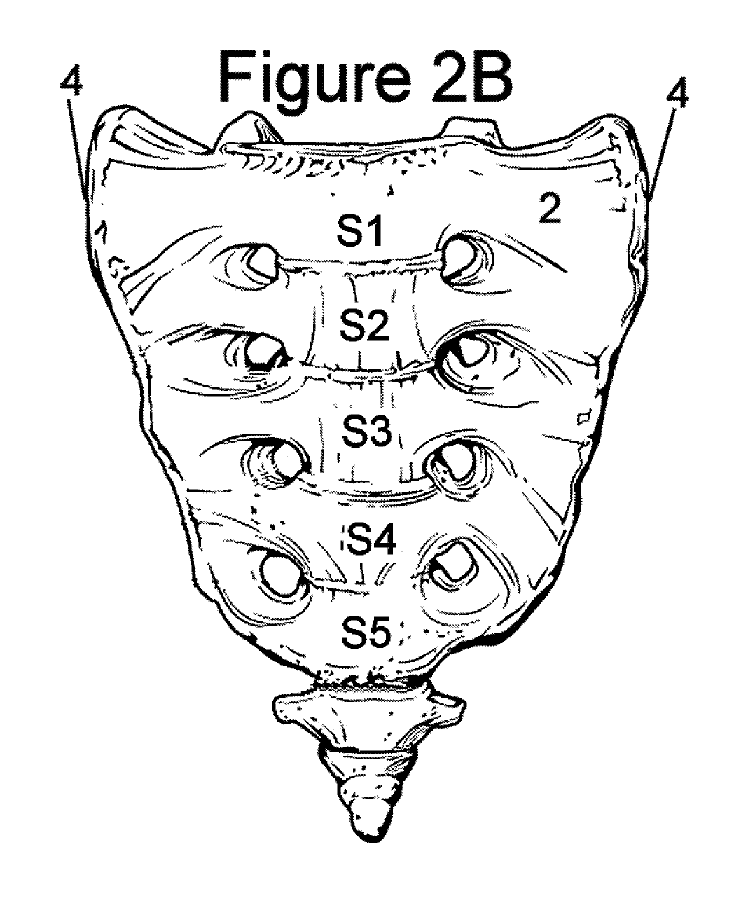

The sacroiliac joint is a diarthrodial joint that joins the sacrum to the ilium bones of the pelvis. In the sacroiliac joint, the sacral surface has hyaline cartilage that moves against fibrocartilage of the iliac surface. FIG. 2A depicts the medial aspect of a left ilium. The surface of the ilium and sacrum that form the sacroiliac joint is also known as the auricular surface 4. The iliosacral screws of the present invention pass from the ilium into the sacrum through this auricular surface 4. FIG. 2B depicts the anterior view of a sacrum. The auricular surface 4 is on either side of the sacrum 2. Among other things, the sacerum contains five vertebral bodies, S1-55. The iliosacral screws pass through the ilium, through the auricular surface 4, and into the S1 vertebral body for the upper screw and the S2 vertebral body for the lower screw.

The method and apparatus for the minimally invasive treatment of unstable pelvic ring injuries with internal posterior iliosacral screw(s) and bone plate(s) will now be described with respect to the figures.

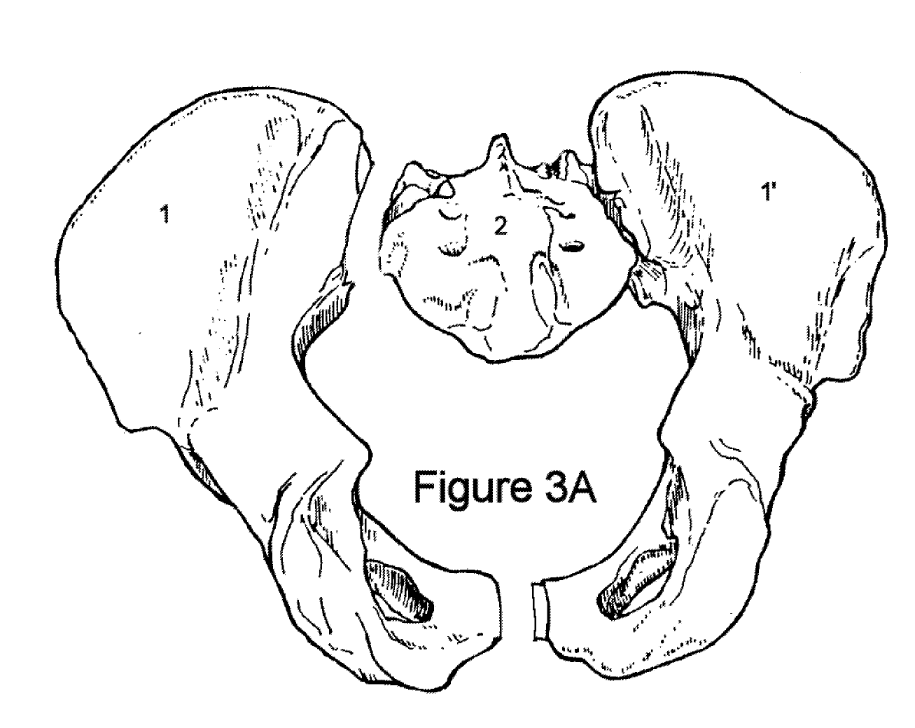

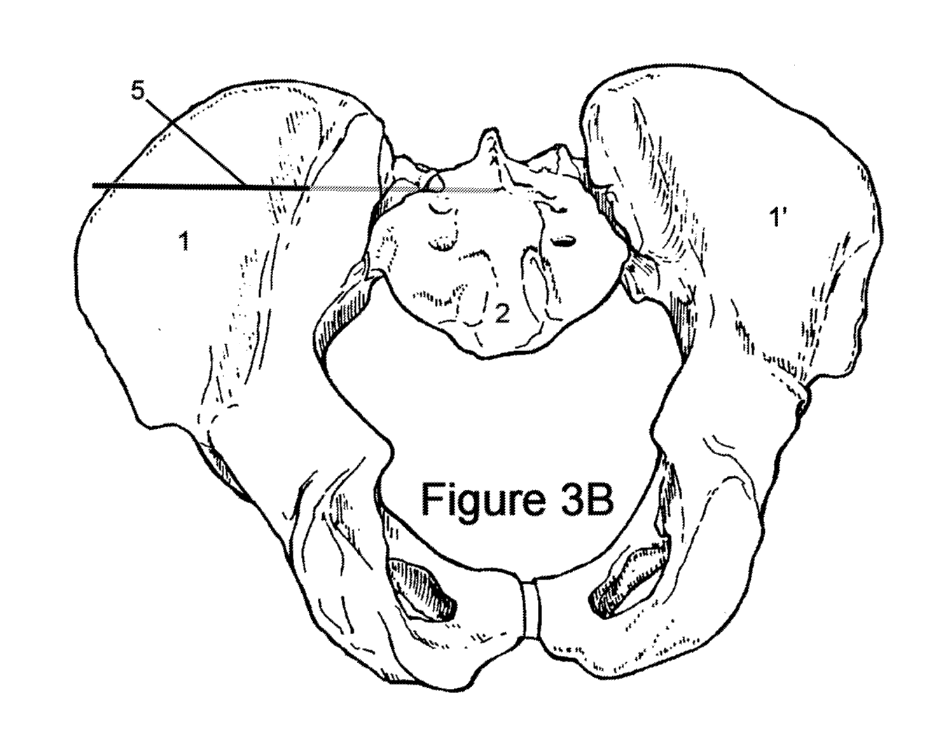

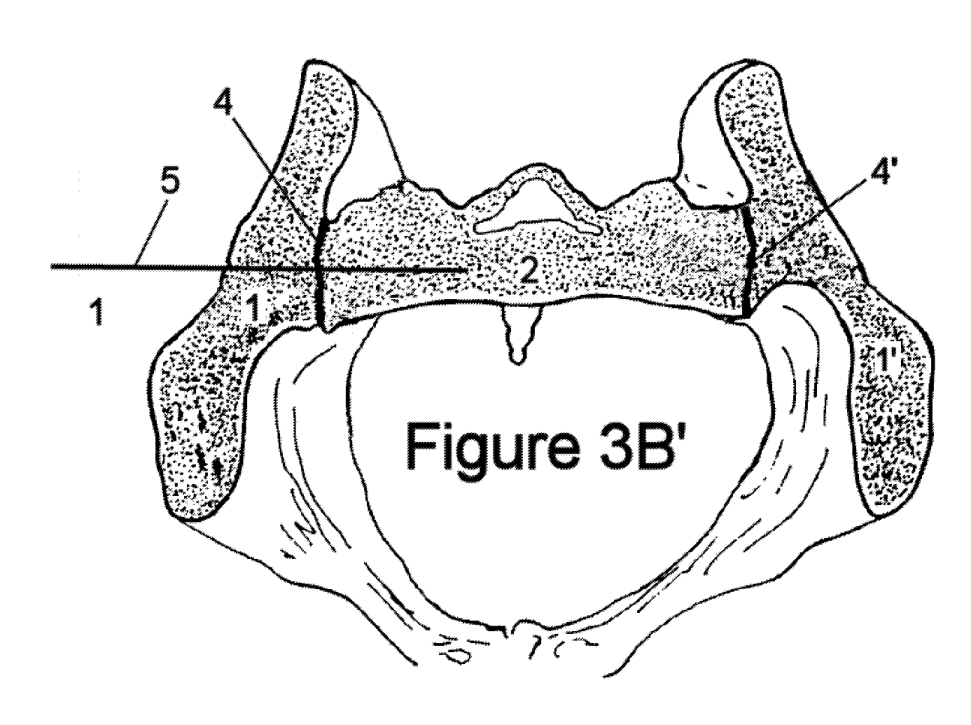

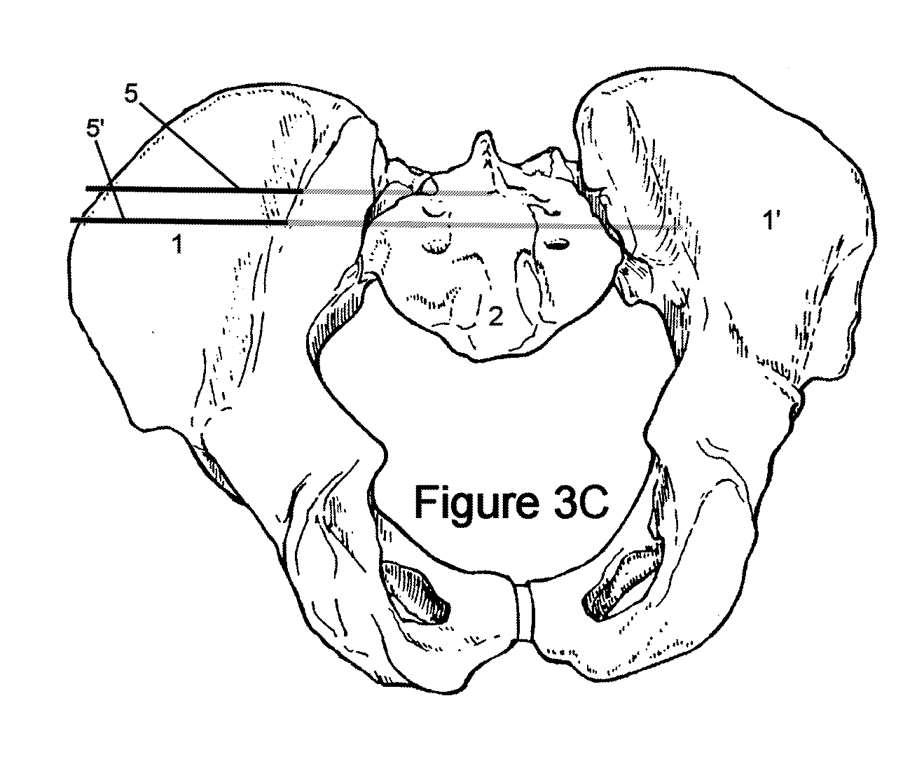

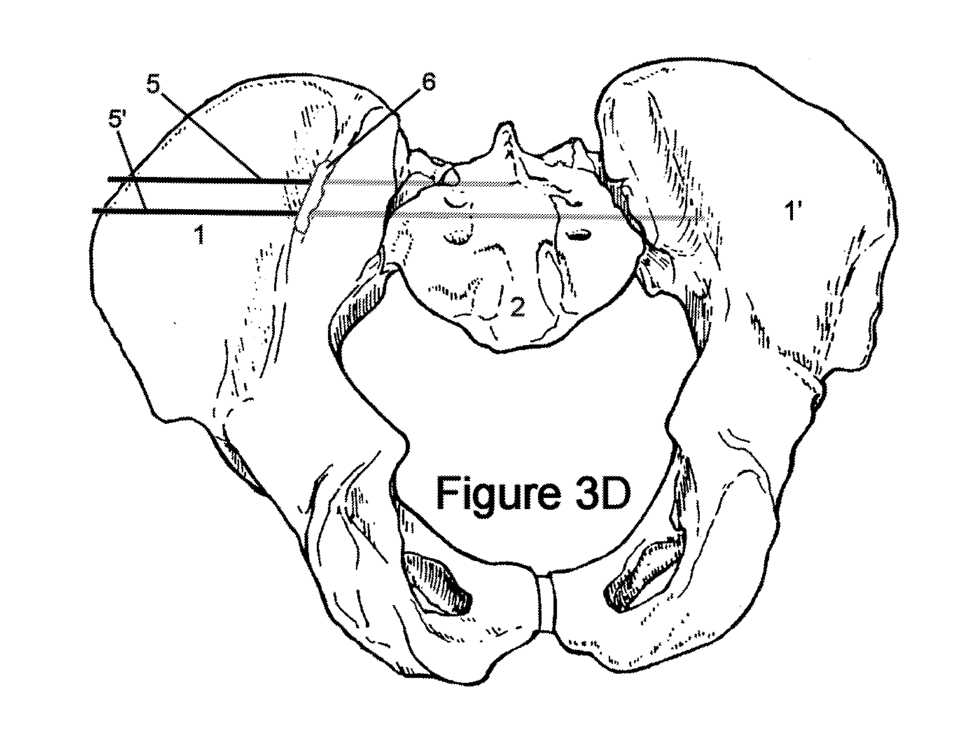

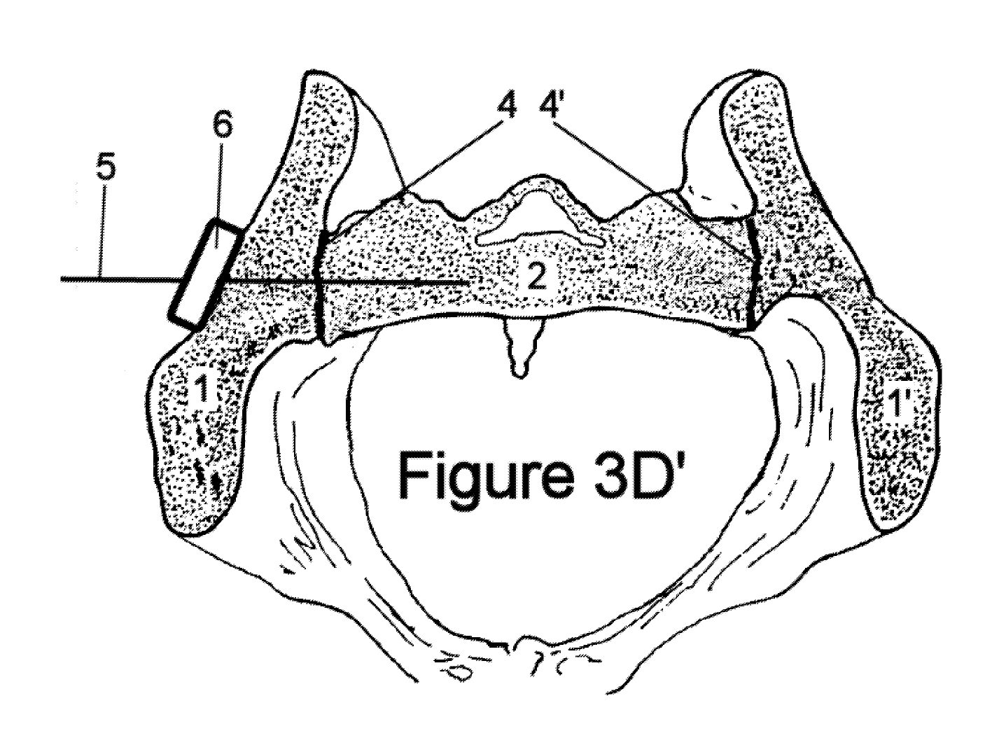

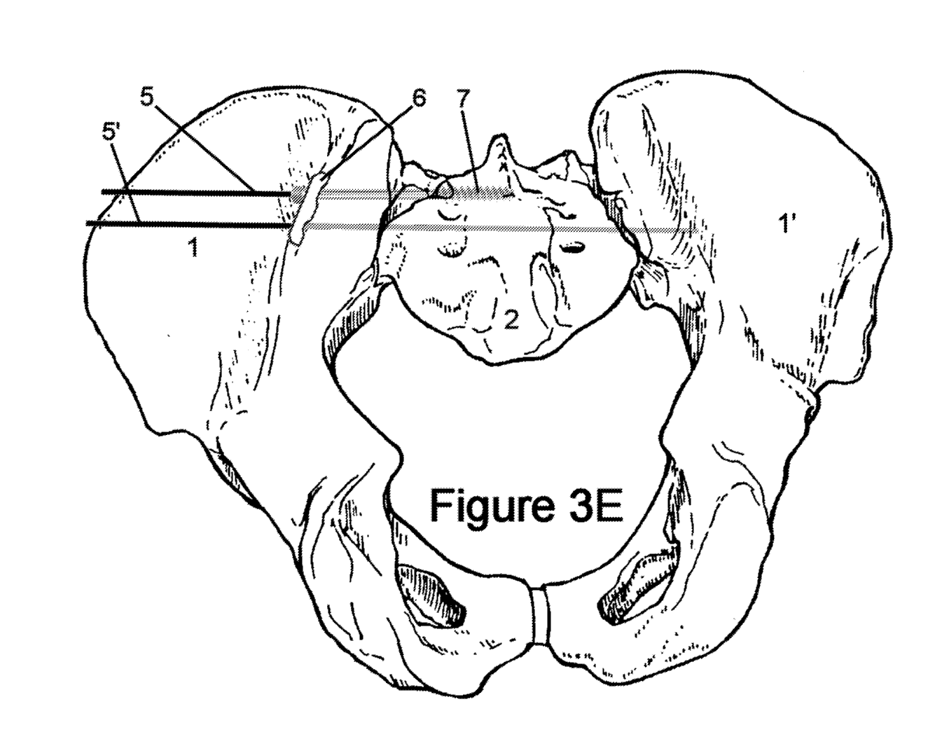

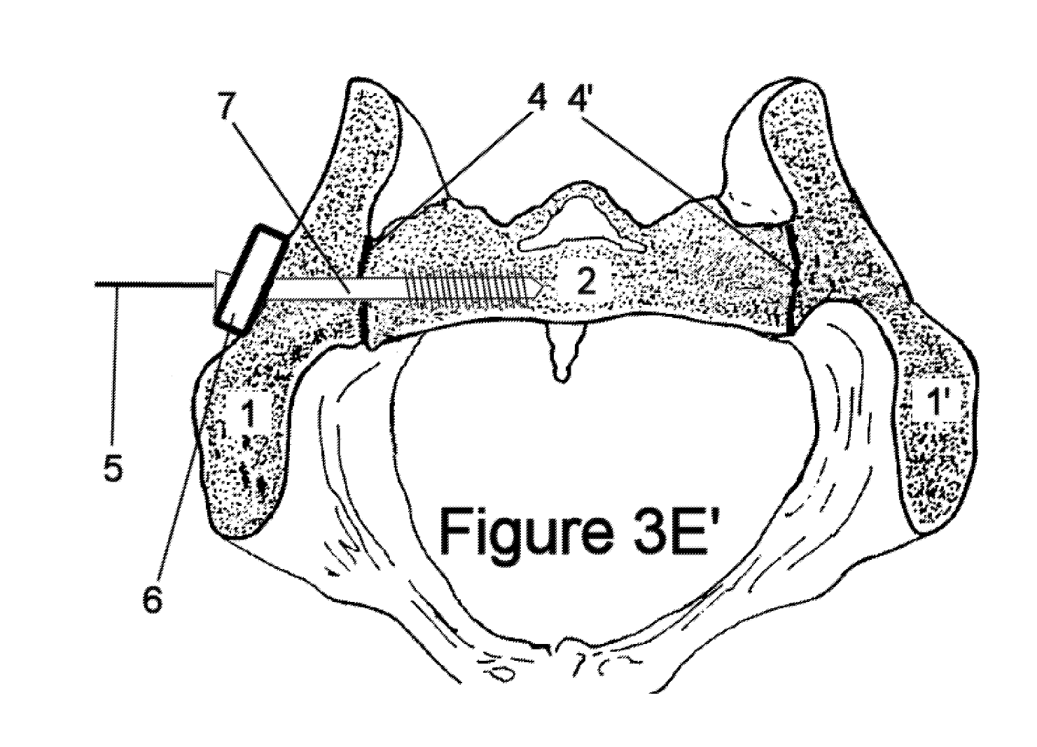

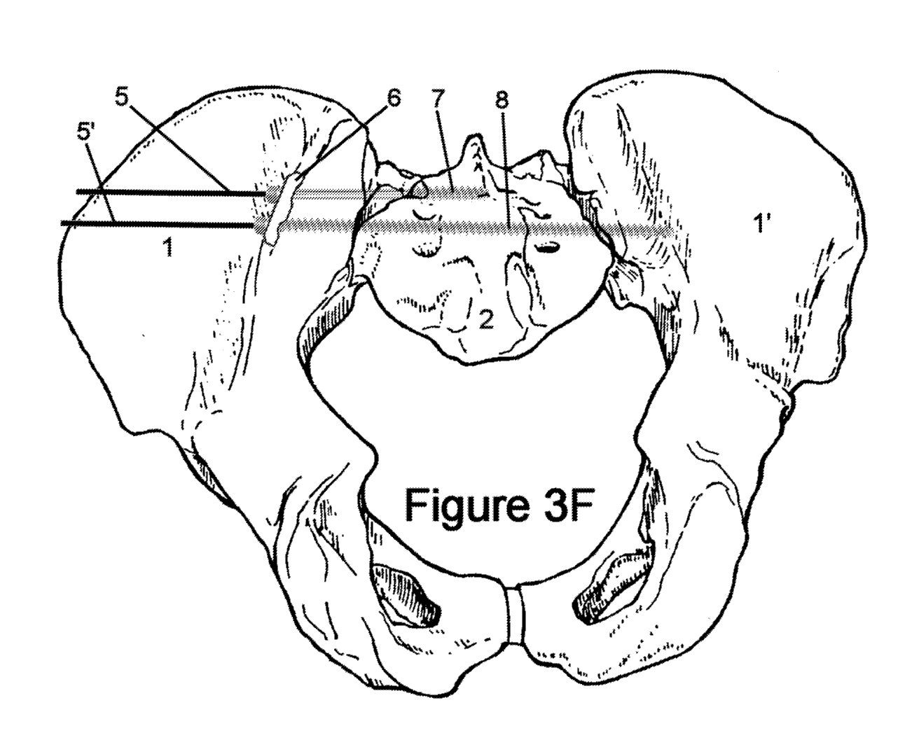

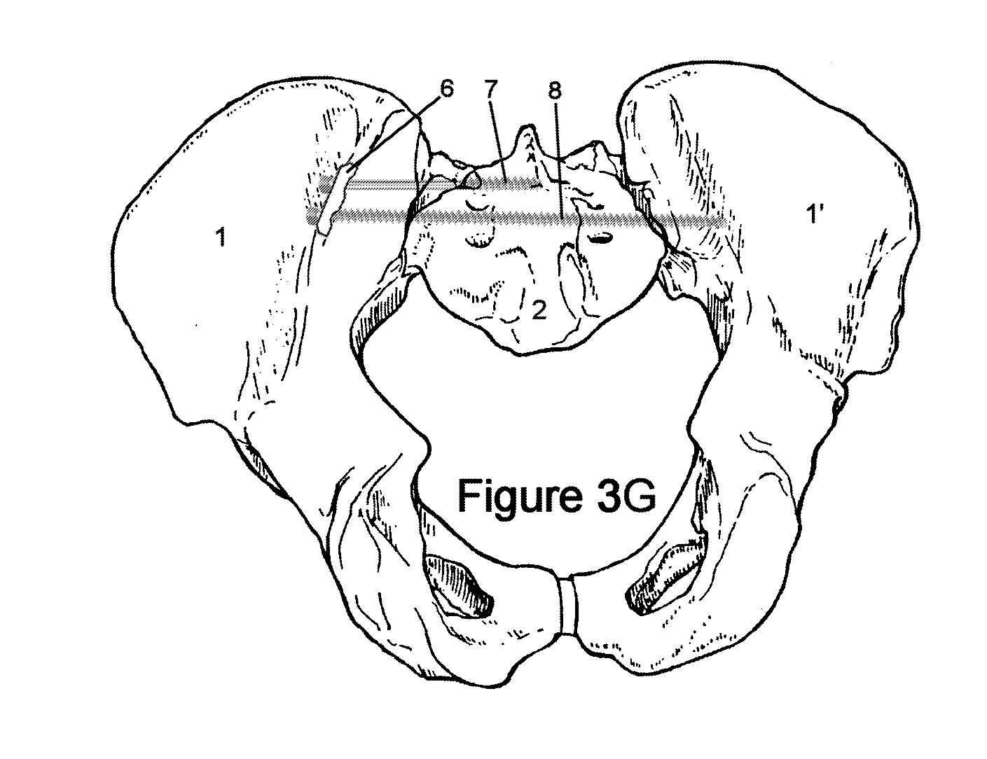

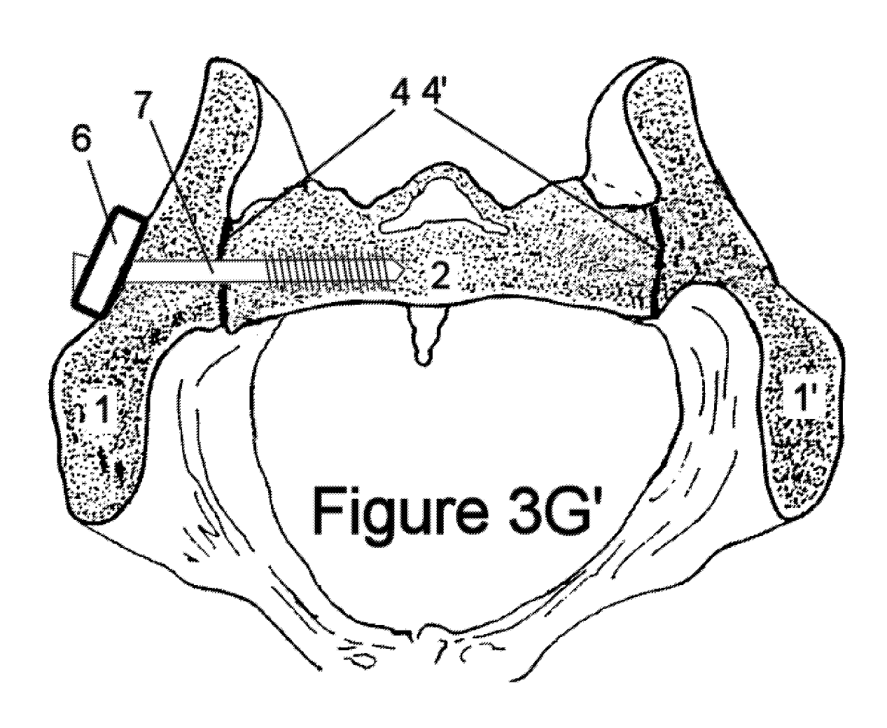

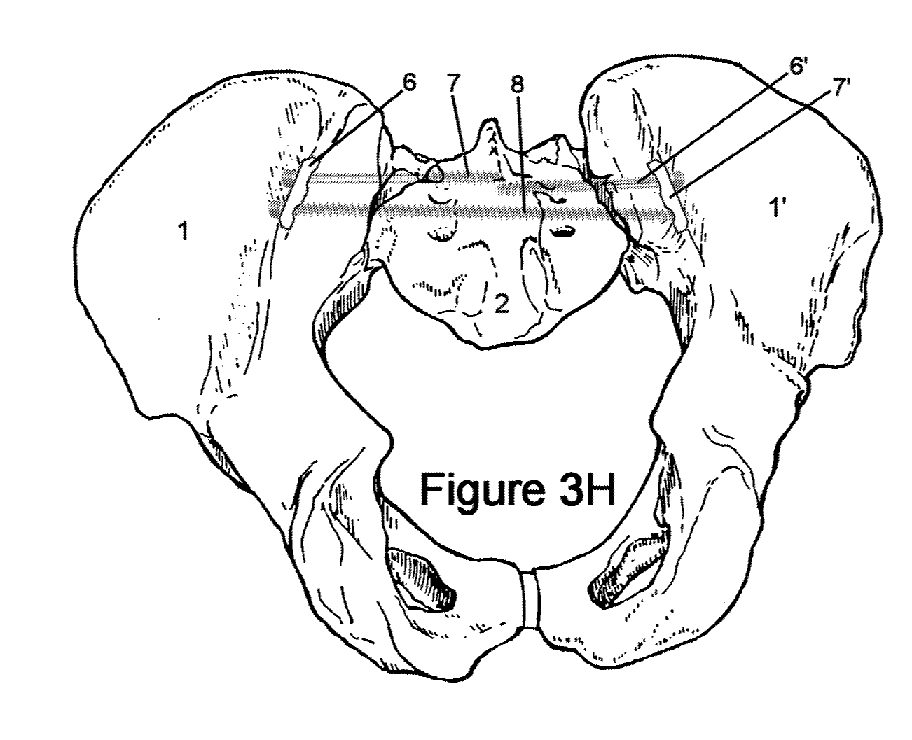

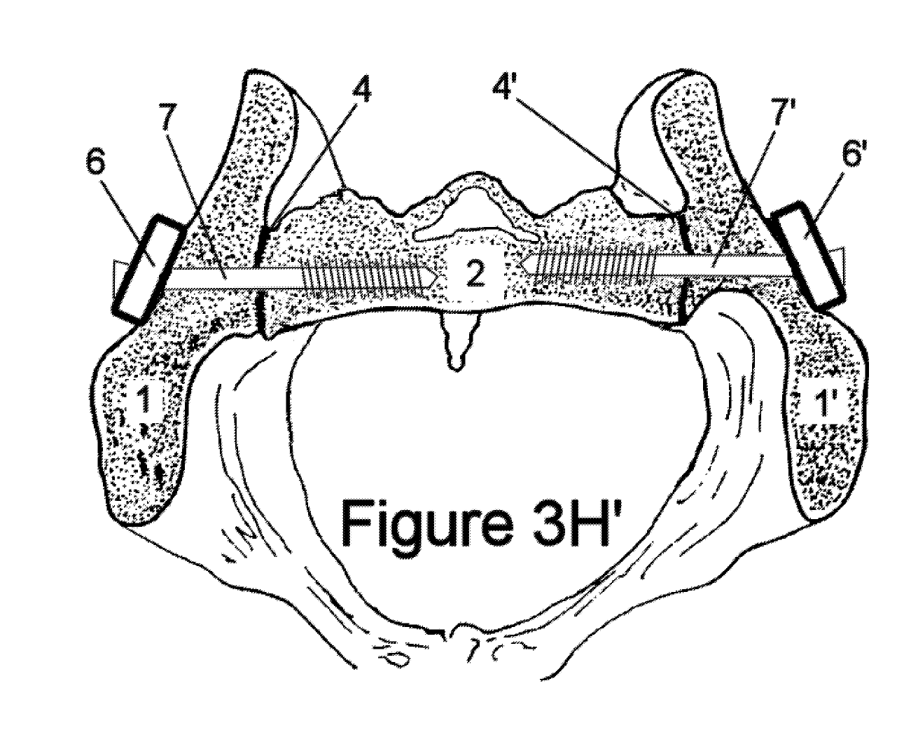

FIG. 3A is a depiction of the posterior of a pelvis having a pelvic ring fracture in which the left ilium 1 has separated from the sacrum 2 and right ilium 1′. FIG. 3B is a depiction of the posterior of a pelvis, wherein the first step of the inventive method has been performed. In the first step, the separated ilium 1 and the remainder of the pelvis are manipulated to bring the auricular surfaces of the separated ilium 1 and the sacrum 2 into alignment. Then a cannulation guide wire 5 is inserted into the posterior of the ilium 1 adjacent to the S1 vertebral body and is then passed through the auricular surface and into the S1 vertebral body. The guide wire 5 should be advanced to near the medial area of the S1 vertebral body. FIG. 3B′ depicts an anterior view of a horizontal cross section of the pelvis at the end of the first step, where the guide wire 5 has been advanced through the ilium 1, through the auricular surface 4 (also known as the sacroiliac joint 3), and into the sacrum.

FIG. 3C is a depiction of the posterior of a pelvis, wherein the second step of the inventive method has been performed. In the second step, another cannulation guide wire 5′ is inserted into the posterior of the ilium 1 adjacent to the S2 vertebral body and is then passed through the sacroiliac joint, into and through the S2 vertebral body and into the opposite ilium 1′. The second guide wire 5′ should be advanced through the S2 vertebral body, and if a second plate is to be placed on the opposite ilium 1′, the guide wire should be advanced completely through the opposite auricular surface and through the opposite ilium 1′. It should be noted that the order of the first and second steps is interchangeable. In practice, in some instances, there may be some advantage to the order in which the cannulation guide wires 5, 5′ are inserted.

FIG. 3D is a depiction of the posterior of a pelvis, wherein the third step of the inventive method has been performed. In the third step a two holed bone plate 6 is slid over the guide wires 5, 5′ and advanced to the surface of the ilium 1. FIG. 3D′ depicts an anterior view of a horizontal cross section of the pelvis at the end of the third step of the inventive method, where the bone plate 6 has been slid over guide wires 5, 5′ and advanced to the surface of the ilium 1.

FIG. 3E is a depiction of the posterior of a pelvis, wherein the fourth step of the inventive method has been performed. In the fourth step a cannulated screw 7 (either cancellous or cortical and either fully or partially threaded) is inserted onto the superior cannulation guide wire 5, and screwed through the superior opening in the bone plate 6, through the ilium 1, the auricular surface and into the S1 vertebral body. Preferably, the cannulated screw 7 is long enough to extend from the bone plate 6, to a medial location within the S1 vertebral body. FIG. 3E′ depicts an anterior view of a horizontal cross section of the pelvis at the end of the fourth step of the inventive method, where the cannulated screw 7 has been screwed through the bone plate 6 and into the S1 vertebral body.

FIG. 3F is a depiction of the posterior of a pelvis, wherein the fifth step of the inventive method has been performed. In the fifth step another cannulated screw 8 is inserted onto the inferior cannulation guide wire 5′, and screwed through the inferior opening in the bone plate 6, through the ilium 1, the auricular surface and the S2 vertebral body. Preferably, the cannulated screw 8 is long enough to extend from the bone plate 6 to a position all of the way through the S2 vertebral body and into the opposite ilium 1′. Again, if a second plate is to be placed on the opposite ilium 1′, the cannulated screw 8 should be advanced completely through the opposite ilium 1′ and protrude far enough that it may be affixed to the second plate. It should be noted that the order of the fourth and fifth steps is interchangeable. In practice, in some instances, there may be some advantage to the order in which the cannulated screws 7, 8 are inserted.

FIG. 3G is a depiction of the posterior of a pelvis, wherein the sixth step of the inventive method has been performed. In the sixth step, the cannulation guide wires 5, 5′ are removed leaving the cannulated iliosacral screws 7, 8 and the bone plate 6 in place to fixate the unstable pelvic ring fracture. FIG. 3G′ depicts an anterior view of a horizontal cross section of the pelvis at the end of the sixth step of the inventive method, where the cannulation guide wires 5, 5′ have been removed leaving the iliosacral screws 7, 8 and the bone plate 6 in place.

FIG. 3H is a depiction of the posterior of a pelvis, wherein optional additional steps have been performed to create a bilateral fixation by adding an additional iliosacral screw 7′ and bone plate 6′ to the opposite iliosacral joint of the pelvis. That is, a cannulation guide wire is inserted through the other ilium 1′, the other auricular surface and into the S1 vertebral body. Another bone plate 6′ is placed over the cannulation guide wire and then an additional cannulated screw 7′ is placed onto the additional cannulation guide wire, and screwed through the superior opening in the bone plate 6′, through the ilium 1′, the auricular surface and into the S1 vertebral body. Preferably, the cannulated screw 7′ is long enough to extend from the bone plate 6′, to a medial location within into the S1 vertebral body. The inferior opening in the bone plate 6′ is affixed to the end of the cannulated screw 8 that protrudes from the opposite ilium 1′. This may be accomplished by either: 1) threading the protruding end of the cannulated screw 8 into the threads of the second bone plate 6′; or, 2) by passing the protruding end of the cannulated screw 8 through the inferior opening in the bone plate 6′ and threading a nut (not shown) onto the protruding end the cannulated screw 8 and tightening the nut against the bone plate 6′. FIG. 3H′ depicts an anterior view of a horizontal cross section of the pelvis at the end of the optional additional steps of the inventive method. As described above an additional cannulated screw 7′ is inserted through the superior opening in an additional bone plate 6′, through the opposite ilium 1′, the opposite auricular surface 4′, and into the S1 vertebral body.

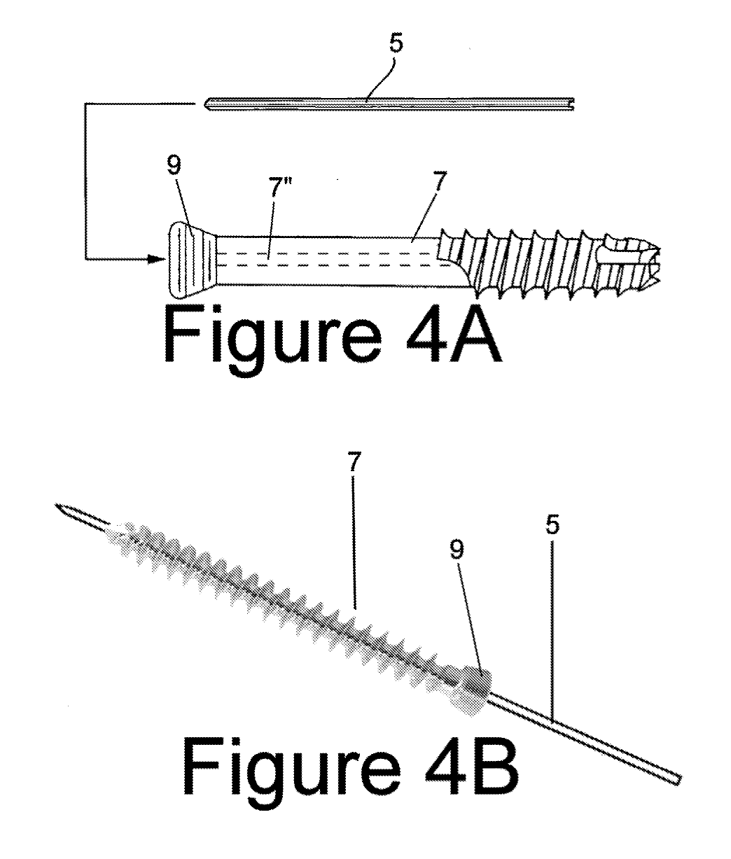

FIG. 4A depicts a cross section of a cannulated screw 7 and specifically shows how a cannulated screw 7 may be inserted onto the cannulation guide wire 5. FIG. 4B show a cannulated screw 7 inserted onto a cannulation guide wire 5. Also shown in both Figures is the locking head 9 of the screw, because the preferred embodiment is used in a variable-angle locked plating system.

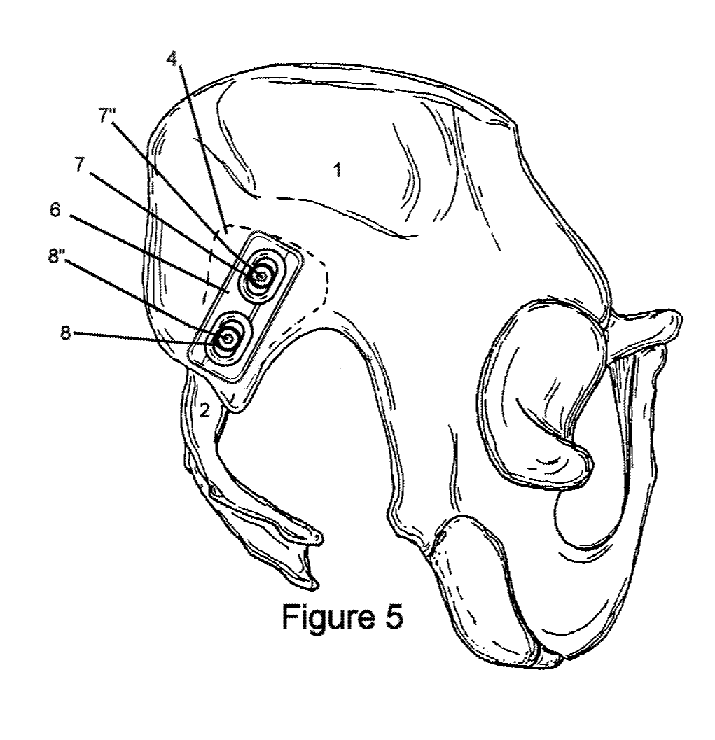

FIG. 5 is a depiction of a lateral view of a pelvis (ilium 1, sacrum 2) having been fixated using the inventive method and apparatus. The cannulated screws 7, 8 pass through the auricular surface 4, holding ilium 1 and sacrum 2 together. The bone plate 6 gives the fixation construct additional strength by resisting the possibility of pulling the cannulated screws 7, 8 through the ilium 1, 1′ when the pelvis is weight bearing. Further, one can see the cannulation openings 7″, 8″ in the cannulated screws 7, 8.

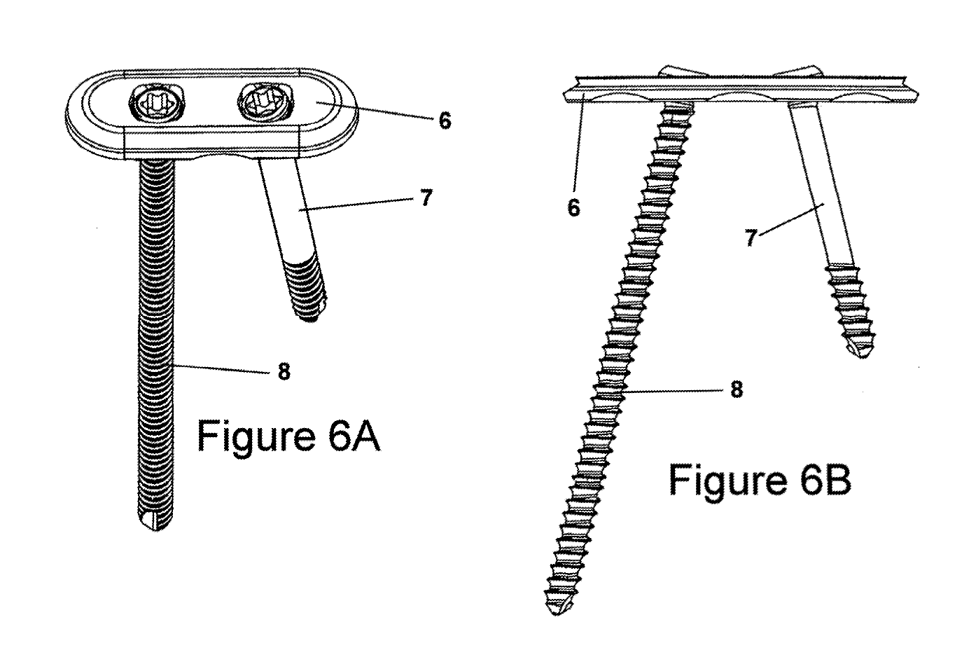

FIGS. 6A and 6B are depictions of different views of a type bone plating system, a variable-angle locked plating system, useful in the present invention. While other bone plating systems may be used, the variable-angle locked plating system is preferred in that this type of system allows for conformity of the bone plate 6, 6′ with the ilium 1, 1′, while allowing the cannulated screws 7, 7′, and 8 to be inserted into the ilium/sacrum at the needed proper angle. The screws and plates of the present invention are formed from sturdy bio-compatible materials, preferably titanium. The screws may be between 7.0-8.5 mm in outer diameter (O.D.), inclusive, and the cannulation guide wires may be 3 mm O.D.

After stabilizing the posterior elements via the iliosacral screws and bone plate(s) method/construct of the present invention, the anterior pelvis may be addressed. Preferably the anterior fixation methods/apparatuses are those disclosed in U.S. Pat. Nos. 8,900,278; 8,814,866; 8,398,635; and 8,177,785, the disclosures of which are herein incorporated by reference.

It is to be expected that considerable variations may be made in the embodiments disclosed herein without departing from the spirit and scope of this invention. Accordingly, the significant improvements offered by this invention are to be limited only by the scope of the following claims.

I claim:

1. A surgical method for minimally invasive affixation of an ilium to the sacrum in an unstable pelvic ring injury, said method comprising the steps of:
providing a bone plate having at least two attachment holes therethrough, and two cannulated screws;
affixing said bone plate to the posterior of said ilium by the steps of:
placing said bone plate on the ilium, adjacent to the sacroiliac joint;
screwing the first of said two cannulated screws through a first of said attachment holes in said bone plate, through the ilium, through the sacroiliac joint, and into the S1 vertebral body; and
screwing the second of said two cannulated screws through a second of said attachment holes in said bone plate, through the ilium, through the sacroiliac joint, and into the S2 vertebral body;
said cannulated screws inserted far enough to hold said bone plate tightly against said ilium.

2. The surgical method of claim 1, wherein said step of placing said bone plate on the ilium, adjacent to the sacroiliac joint comprises the steps of:
inserting a first cannulation guide wire through the ilium, the sacroiliac joint, and into the S1 vertebral body;
inserting a second cannulation guide wire through the ilium, the sacroiliac joint, and into the S2 vertebral body;
sliding said first and second cannulation guide wires through said first and second attachment holes in said bone plate, respectively; and
advancing said bone plate down along said guide wires to the surface of said ilium.

3. The surgical method of claim 2, wherein said step of screwing said first of said two cannulated screws through said first of said attachment holes in said bone plate comprises:
placing said first of said two cannulated screws onto said first cannulation guide wire;
guiding said first of said two cannulated screws down said first cannulation guide wire to the surface of said ilium; and
screwing said first of said two cannulated screws through said ilium, through said sacroiliac joint and into said S1 vertebral body.

4. The surgical method of claim 3, wherein said first of said two cannulated screws is of a length to reach from said bone plate to a medial position in said S1 vertebral body.

5. The surgical method of claim 3, wherein said step of screwing said second of said two cannulated screws through said second of said attachment holes in said bone plate comprises:
placing said second of said two cannulated screws onto said second cannulation guide wire;
guiding said second of said two cannulated screws down said second cannulation guide wire to the surface of said ilium; and
screwing said second of said two cannulated screws through said ilium, through said sacroiliac joint and into said S2 vertebral body.

6. The surgical method of claim 5, wherein said first of said two cannulated screws is of a length to reach from said bone plate to through said S2 vertebral body and into the opposite ilium.

7. The surgical method of claim 5, comprising the further step of removing said first and second cannulation guide wires from the pelvis after insertion of said first and second cannulated screws.

8. The surgical method of claim 1, comprising the further steps of:
placing a second bone plate having at least two attachment holes therethrough on the opposite ilium, adjacent to the opposite sacroiliac joint;
screwing the a third cannulated screw through a first of said attachment holes in said second bone plate, through the opposite ilium, through the opposite sacroiliac joint, and into said S1 vertebral body; and
passing said second cannulated screw through a second of said attachment holes in said second bone plate.

9. The surgical method of claim 8, comprising the further step of screwing threads of the forward end of said second cannulated screw into matching threads in said second of said attachment holes in said second bone plate, thereby locking said second bone plate to said second cannulated screw.

10. The surgical method of claim 8, comprising the further steps of:
passing the forward end of said second cannulated screw through said second of said attachment holes in said second bone plate such that it protrudes from said second of said attachment holes in said second bone plate; and
placing a nut onto the threads of the forward end of said second cannulated screw and tightening said nut to thereby lock said second bone plate to said second cannulated screw.

11. The surgical method of claim 1, wherein said two cannulated screws are cancellous screws, that are fully or partially threaded.

12. The surgical method of claim 11, wherein said first of said two cannulated screws is a partially threaded cancellous screw.

13. The surgical method of claim 11, wherein said second of said two cannulated screws is a fully threaded cancellous screw.

14. The surgical method of claim 1, wherein said bone plate has two attachment holes therethrough, one at either end thereof.

15. The surgical method of claim 8, wherein said second bone plate has two attachment holes therethrough, one at either end thereof.

16. The surgical method of claim 1, wherein said bone plate is a variable-angle locking plate.

17. The surgical method of claim 8, wherein said second bone plate is a variable-angle locking plate.

18. The surgical method of claim 1, wherein said bone plate and said cannulated screws are formed from titanium.

19. The surgical method of claim 1, wherein said cannulated screws are between 7.0 and 8.5 mm in outer diameter, inclusive.

20. The surgical method of claim 2, wherein said first and second cannulation guide wires are about 3 mm in outer diameter.
